(19) United States
(12) Patent Application Publication
(10) Pub. No.: US 2019/0343458 A1
(43) Pub. Date: Nov. 14, 2019

(54) ASSEMBLED HEAD-MOUNTED MODULE FOR SENSING BRAIN ACTIVITY AND HEAD-MOUNTED DEVICE FOR SENSING BRAIN ACTIVITY

(71) Applicant: National Sun Yat-Sen University, Kaohsiung (TW)

(72) Inventor: Yuan-Pin Lin, Kaohsiung (TW)

(21) Appl. No.: 16/408,776

(22) Filed: May 10, 2019

(30) Foreign Application Priority Data
May 11, 2018 (TW) 107206230

(51) Int. Cl. A61B 5/00

(52) U.S. Cl. CPC A61B 5/6803; A61B 5/4064; A61B 5/04008; A61B 5/0082; A61B 5/4836

(57) ABSTRACT

The invention discloses an assembled head-mounted module for sensing brain activity and a head-mounted device for sensing brain activity. The assembled head-mounted module is adapted to be worn on a head of a living body and includes bases and connecting strips. Each base has a hollow portion for accommodating a signal extractor that is configured to extract brain activity signals. The connecting strips are detachably joined to the bases. The bases respectively correspond to portions of the head of the living body suitable for extracting brain activity signals when the assembled head-mounted module is worn on the head of the living body. In the invention, the structure can be flexibly assembled according to practical usage and the components can be joined or disjoined freely, thereby having more flexibility for practical applications.

### RELATED APPLICATIONS

This application claims priority to Taiwan Application Serial Number 107206230, filed on May 11, 2018, which are herein incorporated by reference.

### BACKGROUND

### Technical Field

The present invention relates to an assembled head-mounted module for sensing brain activity and a head-mounted device for sensing brain activity.

### Description of Related Art

With the development of the medical industry and the improvement of health awareness, various types of wearable physiological monitoring devices have been developed in the medical industry, such as head-mounted sensors, smartbelts and smartwatches. On the other hand, with the continuous improvement of the head-mounted brain activity sensing device, the acceptance and practicality of brain signal measurement, brain function diagnosis, brain function stimulation and brain-computer interface applications have been greatly improved. For different brain function applications, the involved brain activities of interest are also different, and thus the number and location of the sensor configuration are also different. If a fixed sensor configuration (the number and/or location of sensors are fixed) is applied, some sensors disposed on some portions of the brain would become redundant, or else the arranged locations of the sensors may not cover the required brain activities of interest. In addition to the above fixed configuration, there is still a configuration method composed of sliding sensors (the number of sensors is fixed but their positions can be slid on route). However, some sliding sensors not on the brain regions of interest remain redundant.

Furthermore, the position of the brain activity sensor needs to be adjusted depending on the shape and size of the head of different subjects. However, the sensor configuration manners upon the fixed or sliding sensors confines the arrangement of the sensor placement. Specifically, the conventional configuration of the brain activity sensing is designed with a fixed number of still sensors, or a fixed number of sliding sensors, making it impossible to flexibly and efficiently configure the sensors on the brain regions of interest only and thus greatly confines the applicability and practicality of the brain activity sensor.

### SUMMARY

The objective of the present invention is to provide an assembled head-mounted module for brain activity sensing and a head-mounted brain activity sensing device, of which the arrangements can be flexibly adjusted according to application requirements and the components can be joined and disjoined freely, thereby having more flexibility for practical applications.

One aspect of the invention relates to an assembled head-mounted module that is adapted to be worn on a head of a living body and includes bases and first connecting strips. Each base has a hollow portion for accommodating a signal extractor to extract brain activity signals. The first connecting strips are detachably joined to the bases. The bases respectively correspond to portions of the head of the living body suitable for extracting brain activity signals when the assembled head-mounted module is worn on the head of the living body.

In accordance with one embodiment of the invention, each base has at least one joining structure adapted to be joined with at least one of the first connecting strips.

In accordance with one embodiment of the invention, the joining structure of each base includes a joining protrusion, a slot, a female buckle, or a combination thereof.

In accordance with one embodiment of the invention, each first connecting strip has at least one joining structure configured to be joined with at least one of the bases.

In accordance with one embodiment of the invention, the joining structure of each first connecting strip includes a joining hole, a tongue, a male buckle, or a combination thereof.

In accordance with one embodiment of the invention, the material of the bases and the joining structures is plastic, resin, or a combination thereof.

In accordance with one embodiment of the invention, the first connecting strips are flexible strips, bendable strips, length-adjustable strips, or a combination thereof.

In accordance with one embodiment of the invention, the assembled head-mounted module further includes at least one second connecting strip detachably joined to at least one of the first connecting strips. The second connecting strip corresponds to at least one ear portion of the living body while wearing the assembled head-mounted module.

In accordance with one embodiment of the invention, the at least one second connecting strip is two second connecting strips, and the second connecting strips respectively correspond to two opposite ear portions of the living body when the assembled head-mounted module is worn on the head of the living body.

In accordance with one embodiment of the invention, the assembled head-mounted module further includes a third connecting strip detachably joined to the second connecting strips. The third connecting strip corresponds to a neck or a jaw of the living body while wearing the head-mounted module.

In accordance with one embodiment of the invention, the third connecting strip is a length-adjustable strip or an elastic strip.

In accordance with one embodiment of the invention, the second connecting strip has a ring shape or has an arc portion.

Another aspect of the invention relates to a head-mounted brain activity sensing device adapted to be worn on a head of a living body and includes first bases, first connecting strips, and one or more signal extractors. The first connecting strips are detachably joined to the first bases. The signal extractors are respectively removably placed in the first bases and are configured to extract brain activity signals. The signal extractors respectively correspond to one or more portions of the head of the living body suitable for extracting brain activity signals when the head-mounted brain activity sensing device is worn on the head of the living body.

In accordance with one embodiment of the invention, the head-mounted brain activity sensing device further includes one or more conductive lines respectively coupled to the signal extractors.

In accordance with one embodiment of the invention, each conductive wire is at least partially encapsulated or covered by the first bases or the first connecting strips.

In accordance with one embodiment of the invention, the head-mounted brain activity sensing device further includes a second base and a breakout box. The second base is detachably joined to one or more of the first connecting strips. The breakout box is removably placed in the second base and coupled to the conductive wires.

In accordance with one embodiment of the invention, the second base corresponds to a posterior portion of the head while wearing the head-mounted brain activity sensing device.

In accordance with one embodiment of the invention, each signal extractor includes a sensor, a probe, or a combination thereof.

In accordance with one embodiment of the invention, the head-mounted brain activity sensing device further includes one or more stimulators removably placed in the first bases. The stimulators are configured to perform brain activity stimulation on the head of the living body.

In accordance with one embodiment of the invention, one of the signal extractors and one of the stimulators are placed in the same one of the first bases.

In the invention, the assembled head-mounted module and the head-mounted brain activity sensing device of the invention allows a plurality of the bases and the connecting strips may be adjusted according to application requirements, and the connecting strips are detachably joined to the bases. Therefore, the assembled head-mounted module and the head-mounted brain activity sensing device of the invention have more flexibility for practical applications and are easier to be received and arranged. Moreover, in comparison with the conventional brain activity sensing device, the assembled head-mounted module and the head-mounted brain activity sensing device of the invention further have the advantage of low manufacturing cost.

### BRIEF DESCRIPTION OF THE DRAWINGS

Embodiments and advantages thereof can be more fully understood by reading the following description with reference made to the accompanying drawings as follows:

FIG. 1 is a perspective view of an assembled head-mounted module for sensing brain activity in accordance with some embodiments of the invention.

FIG. 2 is a schematic diagram in which the assembled head-mounted module of FIG. 1 is worn on a user's head.

FIG. 3 is a schematic diagram of the electrode placement locations of the International 10-20 system.

FIG. 4A is a planar view of the first base of FIG. 1.

FIG. 4B is a planar view of the first connecting strip of FIG. 1.

FIG. 4C is a planar view illustrating that the first base and the first connecting strip of FIG. 1 are joined to each other.

FIG. 4D is a planar view illustrating that a sleeve is mounted around the first base and the first connecting strip of FIG. 1.

FIGS. 5-7 are respectively schematic diagrams of assembled head-mounted modules worn on a user's head for brain activity sensing in accordance with various embodiments of the invention.

FIG. 8 is a partial view of a head-mounted brain activity sensing device 10 in accordance with some embodiments of the invention.

FIG. 9A and FIG. 9B are perspective views of an assembled head-mounted module for brain activity sensing at different viewing angles in accordance with some embodiments of the invention.

### DETAILED DESCRIPTION

The spirit of the disclosure is clearly described hereinafter accompanying with the drawings and detailed descriptions. After realizing preferred embodiments of the disclosure, any persons having ordinary skill in the art may make various modifications and changes according to the techniques taught in the disclosure without departing from the spirit and scope of the disclosure.

It will be understood that, although the terms “first,” “second,” and “third” may be used herein to describe various elements, components, areas and/or portions, these elements, components, areas and/or portions, should not be limited by these terms. These terms are only used to distinguish elements, components, areas and/or portions.

Terms used herein are only used to describe the specific embodiments, which are not used to limit the claims appended herewith. Unless limited otherwise, the term “a,” “an,” “one” or “the” of the single form may also represent the plural form. Further, the spatially relative terms are intended to encompass different orientations of the device in use or operation in addition to the orientation depicted in the figures.

The document may repeat reference numerals and/or letters in the various examples. This repetition is for the purpose of simplicity and clarity and does not in itself dictate a relationship between the various embodiments and/or configurations discussed.

Regarding the term “coupled” used in the following description, it may be used to indicate that two or more elements are in direct physical or electrical contact with each other, or may also mean that two or more elements may not be in direct contact with each other. “Coupled” may still be used to indicate that two or more elements cooperate or interact with each other.

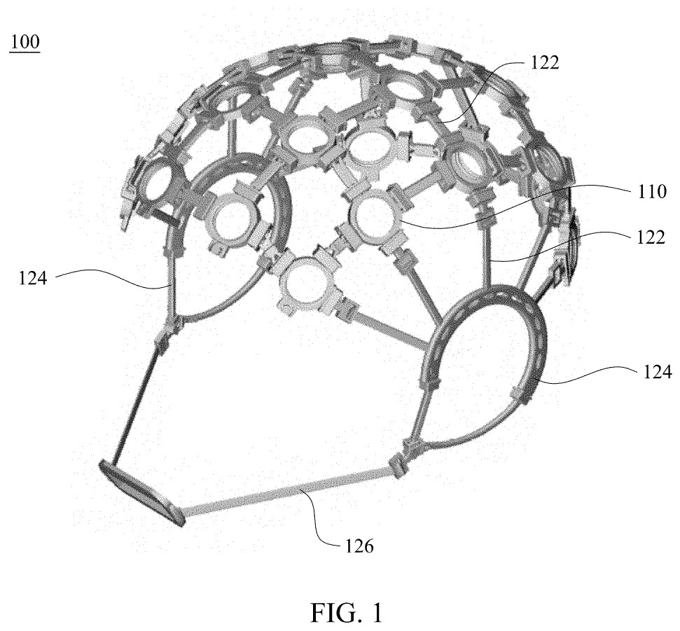

FIG. 1 is a perspective view of an assembled head-mounted module 100 for sensing brain activity in accordance with some embodiments of the invention. As shown in FIG. 1, in the assembled head-mounted module 100, the first bases 110 and the first connecting strips 122 are connected to each other for fixing the relative positions of the first bases 110. The first connecting strips 122 detachably join the first bases 110. That is, the first connecting strips 122 and the first bases 110 can be joined or disjoined to each other freely. The first connecting strips 122 may have different lengths, curvatures and/or degrees of bend, such that the structure of the combination of the first bases 110 and the first connecting strip 122 have a three-dimensional shape. The first connecting strips 122 may be flexible strips, bendable strips, length-adjustable strips, or a combination thereof. In practical applications, the first connecting strip 122 may be properly selected according to the size and the shape of a living body's head, in order to have a better contact of the first bases 110 to the living body's head when the assembly structure of the first bases 110 and the first connecting strips 122 is worn on head, and as such, the quality of the signal extractors placed into the first bases 110 for detecting brain activity can be improved.

In some embodiments, the assembled head-mounted module 100 further includes the second connecting strips 124 that connect one or more first connecting strips 122. The second connecting strips 124 detachably join the first connecting strips 122. That is, the second connecting strips 124 and the first connecting strips 122 can be joined or disjoined to each other freely. The second connecting strips 124 may have ring shapes or have arc portions. As such, the first bases 110, the first connecting strip 122 and the second connecting strips 124 are joined. When the assembled head-mounted module 100 is worn on a living body's head, the second connecting strips 124 correspond to the living body's ear portions, in order to further fix and prevent the assembled head-mounted module 100 from loosing or even falling due to a living body's action. In addition, the number of the second connecting strips 124 may be one or two, which may correspond to the left ear portion, the right ear portion or both of the living body.

Further, in some embodiments, the number of the second connecting strip 124 is two, and the assembled head-mounted module 100 further includes a third connecting strip 126 of which the two ends respectively connects to the second connecting strips 124. The third connecting strip 126 is detachably joined to the second connecting strips 124. That is, the third connecting strip 126 and the second connecting strips 124 can be joined and disjoined to each other freely. The third connecting strip 126 may be a length-adjustable strip or an elastic strip. As such, when the assembled head-mounted module 100 is worn on a living body's head, the third connecting strip 126 corresponds to the living body's jaw portion, in order to further fix and prevent the assembled head-mounted module 100 from loosing or even falling during body motion. In other embodiments, the number of the third connecting strip 126 may be two. One end of the third connecting strips 126 are respectively connected to the second connecting strips 124, and the other end of the third connecting strips 126 may respectively have corresponding buckle and buckle base. When the assembled head-mounted module 100 is worn on a living body's head, the engagement of the buckle and the buckle base of the third connecting strips 126 may further fix the assembled head-mounted module 100 on the living body's head. The assembled head-mounted module 100 can be removed from the living body's head by separating the buckle and the buckle base of the third connecting strips 126.

Each first base 110 may accommodate a signal extractor for extracting brain activity signals. As such, the assembled head-mounted module 100 and the signal extractors form a head-mounted brain activity sensing device. When the head-mounted brain activity sensing device is worn on a living body's head, the first bases 110 respectively correspond to portions of the living body's head for extracting brain activity signals, and the signal extractors also respectively correspond to the portions suitable for extracting brain activity signals because they are respectively placed in the first bases 110. The signal extractors are removably placed in the first bases 110. Before the head-mounted brain activity sensing device is used, one or more signal extractors may be respectively placed into one or more first bases 110 according to application requirements; after the head-mounted brain activity sensing device is used, the signal extractors may be removed from the first bases 110. In addition, each signal extractor may include a sensor (for electrical sensing, optical sensing and/or magnetic sensing), a probe or a combination thereof, but is not limited thereto.

In practical applications, one or more signal extractors may be respectively placed into one or more first bases 110 of the assembled head-mounted module 100 to form a head-mounted brain activity sensing device before or after wearing the head-mounted brain activity sensing device on a user's head H. Alternatively, the assembled head-mounted module 100 may be worn on a user's head H first, and then one or more signal extractors may be respectively placed into one or more first bases 110 of the assembled head-mounted module 100 to form a head-mounted brain activity sensing device.

In some embodiments, each first base 110 may accommodate a stimulator (e.g. an electrode, an electrode array or another suitable component) for performing brain activity stimulation on living body's head, such as by electrical stimulation and/or magnetic stimulation. In various embodiments, each first base 110 may accommodate a signal extractor, a stimulator, or a combination thereof. That is, one or more first bases 110 of the assembled head-mounted module 100 may accommodate signal extractors, other one or more first bases 110 of the assembled head-mounted module 100 may accommodate stimulators, and/or further one or more first bases 110 of the assembled head-mounted module 100 may accommodate both signal extractors and stimulators. As such, the head-mounted brain activity sensing device provides brain-activity sensing (e.g. electrical, optical, or magnetic sensing) and stimulation (e.g. electrical or magnetic stimulation) independently, interactively or simultaneously on the brain regions of interest according to application requirements.

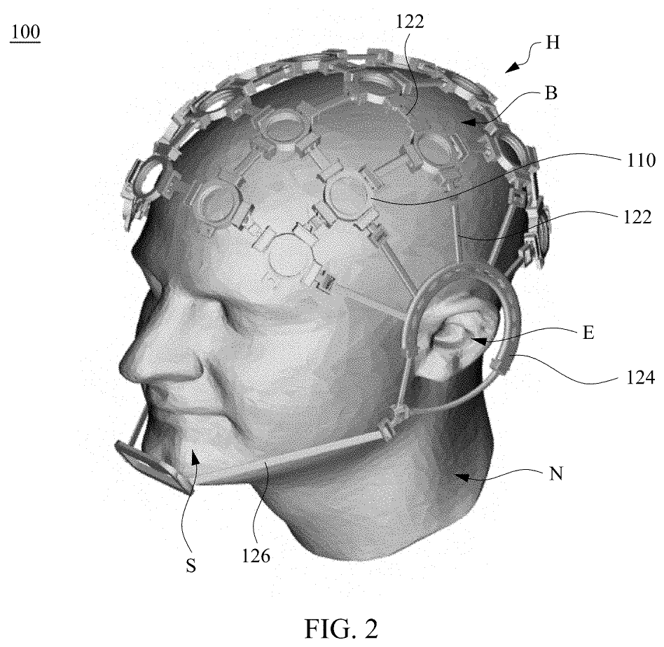

Taking the assembled head-mounted module 100 worn on a human's head for illustration, referring to FIG. 2, which is a schematic diagram in which the assembled head-mounted module 100 of FIG. 1 is worn on a user's head H. As shown in FIG. 2, when the assembled head-mounted module 100 is worn on the user's head H, the first bases 110 and the first connecting strips 122 correspond to the user's brain portion B, the second connecting strips 124 correspond to the user's ear portions E, and the third connecting strip 126 corresponds to the user's jaw portion S.

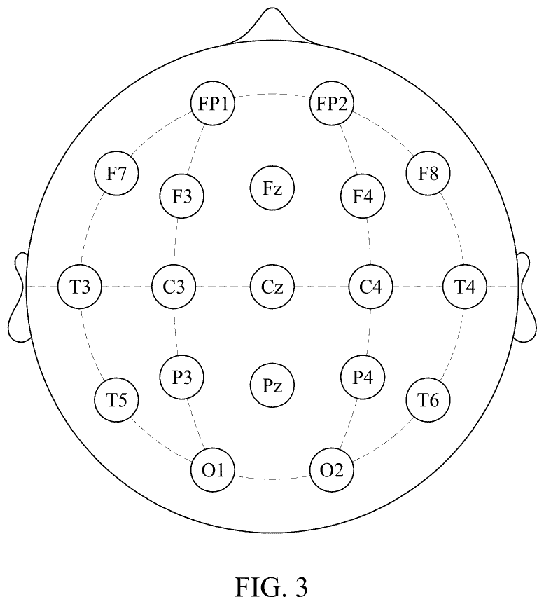

The portions of the first bases 110 on the user's head H may correspond to the electrode placement locations according to the International 10-20 System, the International 10-10 System or another system. FIG. 3 is a schematic diagram which exemplarily illustrates the electrode placement locations of the International 10-20 system. When the assembled head-mounted module 100 is worn on the user's head H, the first bases 110 may correspond to the electrode placement locations Fp1, Fp2, F7, F3, Fz, F4, F8, T3, C3, Cz, C4, T4, T5, P3, Pz, P4, T6, O1, and O2.

In various embodiments, the number of the first bases 110 may be identical to or different from the number of electrode placement locations shown in FIG. 3. For audiometry, the assembled head-mounted module 100 may be worn on the user's head H first, and then a suitable number of the signal extractors are respectively placed in the first bases 110 corresponding to the electrode placement locations F3, F4, T3, C3, Cz, C4, T4, O1, O2, so as to extract brain activity signals during an audiometry testing on the user. Alternatively, a suitable number of the first bases 110 and a suitable number of the first connecting strip 122 with suitable lengths, curvatures and/or degrees of bend forms the assembled head-mounted module 100, such that the first bases 110 respectively correspond to the electrode placement locations F3, F4, T3, C3, Cz, C4, T4, O1, and O2 when the assembled head-mounted module 100 is worn on the user's head H. The signal extractors are then respectively placed in the first bases 110 for extracting brain activity signals during an audiometry testing on the user. It is noted that the aforementioned placement is only for illustration and is not limiting the scope of the invention. An operator may flexibly assemble the number of the signal extractors and configure their placement locations according to application requirements.

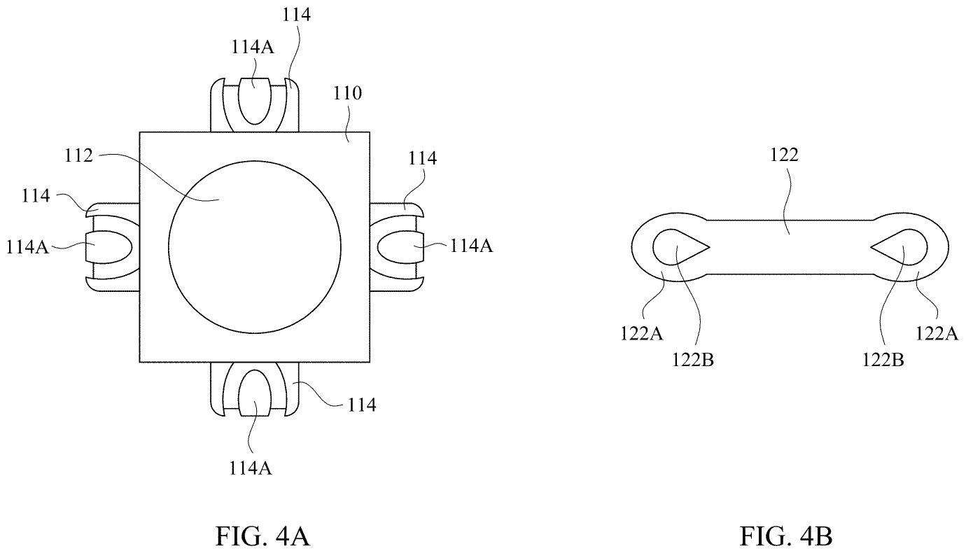

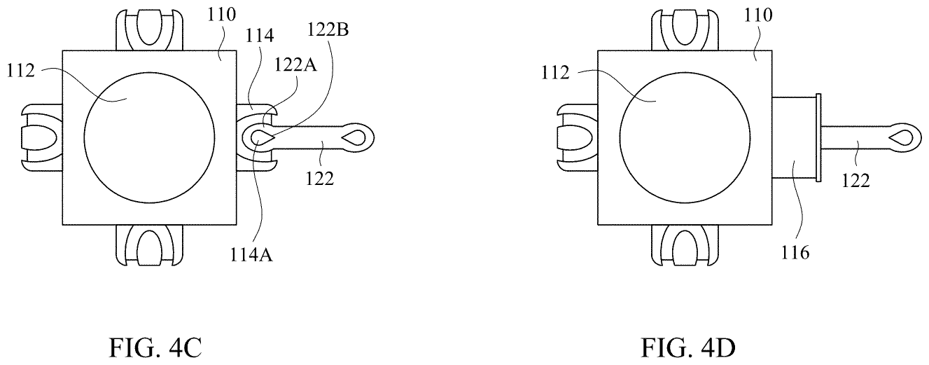

FIG. 4A and FIG. 4B are planar views of the first base 110 and the first connecting strip 122 of FIG. 1, respectively. As shown in FIG. 4A, the first base 110 is square, a hollow portion 112 is at the center of the first base 110, and joining structures 114 are respectively at the side edges of the first base 110. The hollow portion 112 is suitable for accommodating a signal extractor including, but not limited to, a sensor (for electrical sensing, optical sensing and/or magnetic sensing), a probe, or a combination thereof, and the joining structure 114 is suitable for joining the joining structure 122A of the first connecting strip 122 shown in FIG. 4B. According to design and application requirements, the joining structure 114 and the first connecting strip 122 may be joined to each other by, for example, mortising, engaging, plugging, locking or another suitable method. In other embodiments, a stimulator, such as an electrode, an electrode array or another suitable component, may be placed in the hollow portion 112 to perform brain function stimulation on the living body's head. In some embodiments, the hollow portion 112 is adapted to place both a signal extractor and a stimulator therein.

The schematic diagrams illustrated in FIG. 4A and FIG. 4B are exemplified by plugging. As shown in FIG. 4A and FIG. 4B, the joining structures 114, 122A have joining protrusion 114A and joining hole 122B, respectively. As shown in FIG. 4C, the first base 110 and the first connecting strip 122 are joined to each other by plugging the joining protrusion 114A into the joining hole 122B. In some embodiments, as shown in FIG. 4D, after the joining protrusion 114A is plugged in the joining hole 122B, a sleeve 116 may be further mounted around the joining structure 114 for encapsulating the joining protrusion 114A and the joining hole 122B, so as to fasten the first base 110 and the first connecting strip 122 and prevent them from loosing.

It is noted that the joining method of the first base 110 and the first connecting strip 122 illustrated in FIG. 4A to FIG. 4D is only an embodiment of the invention and is not used to limit the scope of the invention. For example, in other embodiments, the joining structures 114, 122A may be corresponding female buckle and male buckle, and the first base 110 and the first connecting strip 122 are joined to each other by inserting the male buckle into the female buckle, or alternatively, the joining structures 114, 122A may be corresponding slot and tongue, the first base 110 and the first connecting strip 122 are joined to each other by engaging the tongue to the slot.

In addition, although the number of the joining structures 114 in the first base 110 shown in FIG. 4A is 4, and the number of the joining structures 122A of the first connecting strip 122 shown in FIG. 4B is 2, the invention is not limited thereto. For example, the number of the first bases 110 and the number of the joining structures 114 may be all 1, 2 or 3, and the number of the joining structures 122A of the first connecting strip 122 may be 3 or 4, which can be correspondingly provided according to application requirements. In addition, the shape of the first bases 110 may be other than square, such as triangular and/or hexagonal, according to application requirements.

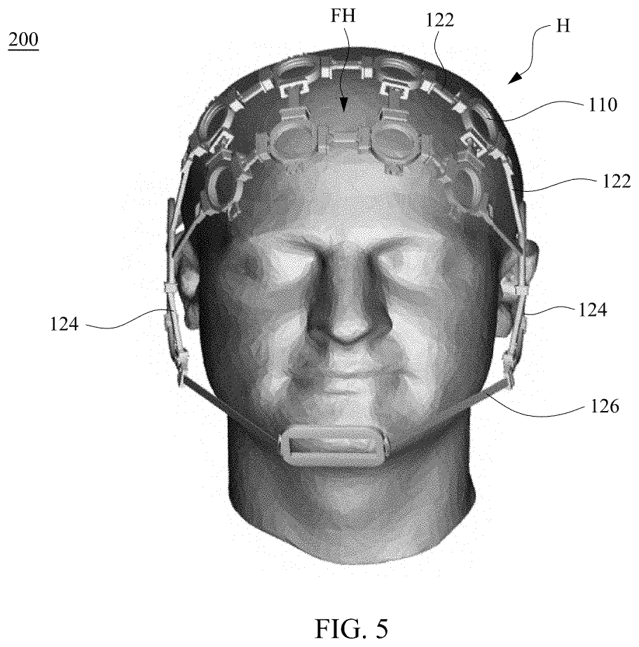

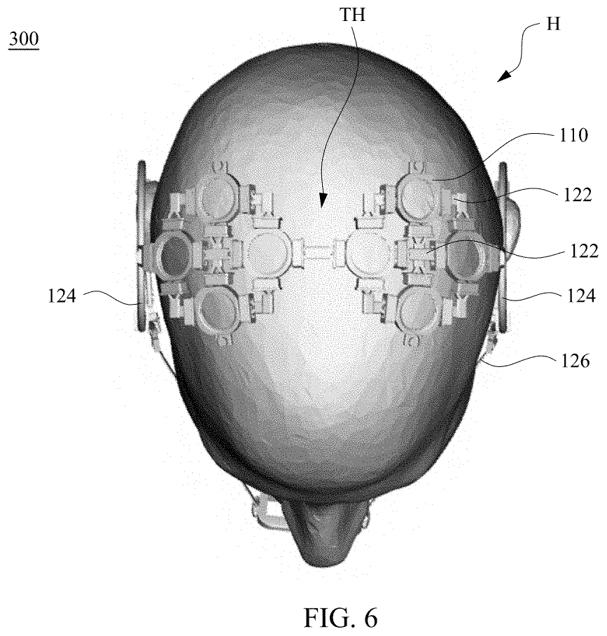

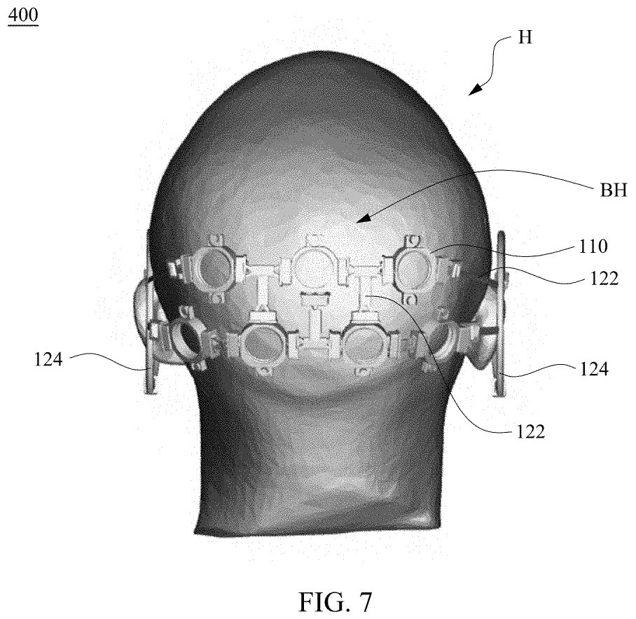

The number of the first bases 110 and the number of the first connecting strips 122 shown in FIG. 1 are only illustrated as examples. In practical applications, the number of the first bases 110 and/or the number of the first connecting strips 122 may be adjusted according to requirements of usage. FIGS. 5-7 are respectively schematic diagrams of assembled head-mounted modules 200, 300, 400 worn on a user's head H for brain activity sensing in accordance with various embodiments of the invention. Similar to the assembled head-mounted module 100 of FIG. 1, each of the assembled head-mounted modules 200, 300, 400 is formed of the first bases 110, the first connecting strips 122, the second connecting strips 124 and the third connecting strips 126. The difference between the assembled head-mounted modules 200, 300, 400 and the assembled head-mounted module 100 of FIG. 1 is that when the assembled head-mounted module 200 is worn on the user's head H, the first bases 110 and the first connecting strips 122 all correspond to the forehead FH (over the frontal lobe of the brain) to measure the brain activity associated more with cognitive function; when the assembled head-mounted module 300 is worn on the user's head H, the first bases 110 and the first connecting strips 122 all correspond to the top of the head TH (over the centro-parietal lobe of the brain) to measure the brain activity associated with sensory and action; when the assembled head-mounted module 400 is worn on the user's head H, the first bases 110 and the first connecting strips 122 all corresponds to the back of the head BH (over the occipital lobe of the brain) to measure the brain activity associated with vision.

As can be seen in FIG. 5 to FIG. 7, the assembly structure of the assembled head-mounted module can be flexibly adjusted according to requirements of usage, and suitable first bases 110 and the first connecting strips 122 are selected, such that the first bases 110 and the first connecting strips 122 correspond to a distinct area of the head H when the assembled head-mounted module is worn on the user's head H. Then, the signal extractors placed in the first bases 110 extract brain activity signals at the distinct area.

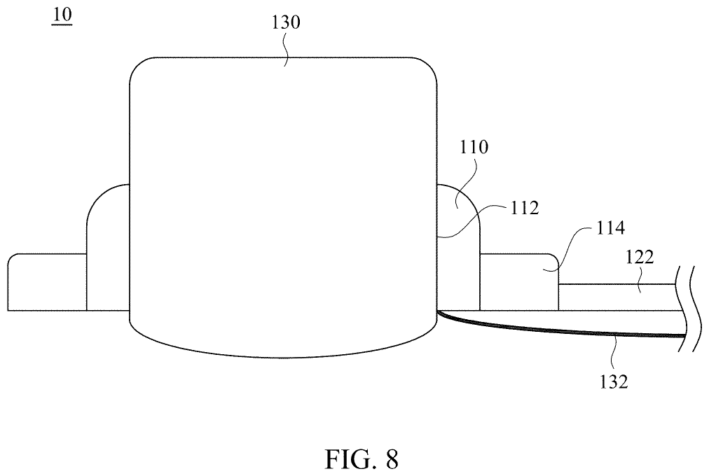

FIG. 8 is a partial view of a head-mounted brain activity sensing device 10 in accordance with some embodiments of the invention. The head-mounted brain activity sensing device 10 may be formed of, for example, the assembled head-mounted module 100, 200, 300, 400, or another assembled head-mounted module mainly formed of the first bases 110 and the first connecting strips 122. For the convenience of description, FIG. 8 illustrates only a single first base 110 and a single first connecting strip 122, but the other schematic diagram of the first base 110 and the first connecting strip 122 may be identical to or similar to that shown in FIG. 8. As shown in FIG. 8, in the head-mounted brain activity sensing device 10, the first connecting strip 122 is joined to the joining structure 114 of the first base 110, while the signal extractor 130 is placed in the hollow portion 112 of the first base 110. The signal extractor 130 may include a sensor (for electrical sensing, optical sensing and/or magnetic sensing), a probe, a combination thereof or another component suitable for extracting brain activity signals. The signal extractor 130 may be further coupled to the conductive wire 132 for transmitting extracted brain activity signals therethrough.

Moreover, for aesthetic appeal and convenient arrangement, the conductive wire 132 may be at least partially encapsulated or covered by the first base 110 and/or the first connecting strip 122. For example, the first base 110 and/or the first connecting strip 122 may have a fixing/hollow structure which can fix/encapsulate the conductive wire 132 under the first base 110 and/or the first connecting strip 122.

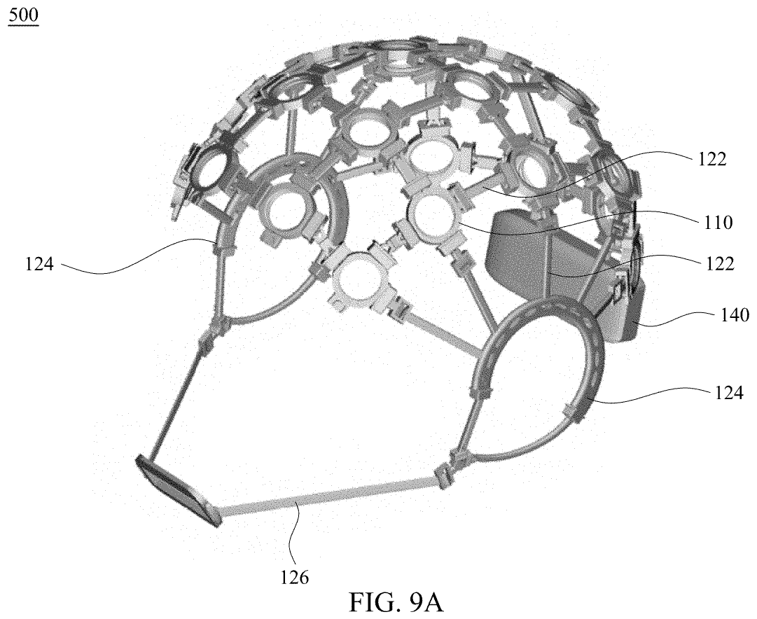

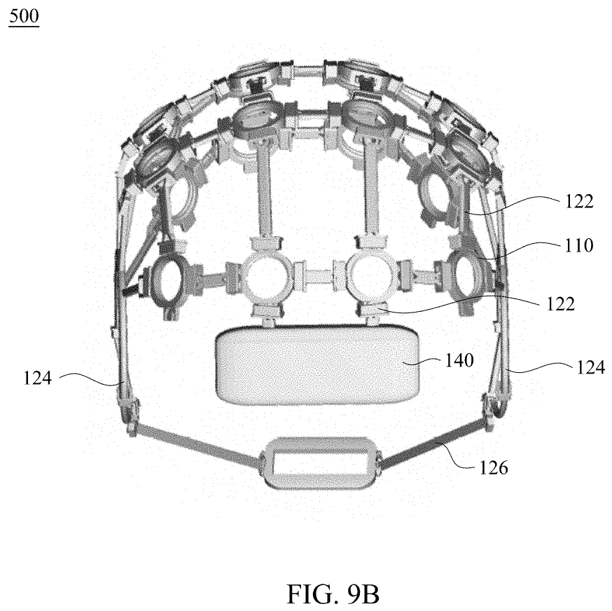

FIG. 9A and FIG. 9B are perspective views of an assembled head-mounted module 500 for brain activity sensing at different viewing angles in accordance with some embodiments of the invention. The assembled head-mounted module 500 is similar to the assembled head-mounted module 100 in FIG. 1, the difference between the assembled head-mounted modules 100, 500 is that the assembled head-mounted module 500 further includes a second base 140 that is detachably joined to one or more first connecting strips 122. The second base 140 may be arranged at the back side of the assembled head-mounted module 500, such that the second base 140 corresponds to the living body's posterior portion while wearing on a living body's head. In some embodiments, the first connecting strips 122 may be connected between the second connecting strips 124 and the second base 140 for enhancing joining strength of the second base 140.

The second base 140 may accommodate a breakout box which is coupled to one or more signal extractors (e.g. through the conductive wire 132 in FIG. 8) and can be used to sense brain activity signals and further amplify and digitize the brain activity signals. The breakout box may further transmit the processed brain activity signals to a computer or a server with brain activity signal process and analysis ability through a wired or wireless communication method. The assembled head-mounted module 500, the signal extractors placed in the first bases 110 and the breakout box placed in the second base 140 forms a head-mounted brain activity sensing device.

In practical applications, one or more signal extractors and a breakout box may be respectively placed in the first bases 110 of the assembled head-mounted module 500 to form a head-mounted brain activity sensing device first, and then the head-mounted brain activity sensing device is worn on a user's head H. Alternatively, the assembled head-mounted module 100 may be worn on a user's head H first, and then one or more signal extractors may be respectively placed into one or more first bases 110 of the assembled head-mounted module 100 to form a head-mounted brain activity sensing device. In the embodiments of the invention, the first bases 110, the sleeve 116, the first connecting strips 122, the second connecting strips 124, the third connecting strip 126 and the second base 140 may be electrically insulating, so as to avoid interfering extracted brain activity signals, thus improving the sensing quality. Their materials can be, but not limited to, plastic, resin and ceramic.

In sum, the assembled head-mounted module and the head-mounted brain activity sensing device of the invention allows a plurality of the bases and the connecting strips may be adjusted according to application requirements, and the connecting strips are detachably joined to the bases. Therefore, the assembled head-mounted module and the head-mounted brain activity sensing device of the invention have more flexibility for practical applications and are easier to be received and arranged. Moreover, in comparison with the conventional brain activity sensing device, the assembled head-mounted module and the head-mounted brain activity sensing device of the invention further have the advantage of low manufacturing cost.

Although the invention is described above by means of the implementation manners, the above description is not intended to limit the invention. A person of ordinary skill in the art can make various variations and modifications without departing from the spirit and scope of the invention, and therefore, the protection scope of the invention is as defined in the appended claims.

What is claimed is:

1. An assembled head-mounted module adapted to be worn on a head of a living body and comprising:
a plurality of bases, each of the bases having a hollow portion for accommodating a signal extractor to extract brain activity signals; and
a plurality of first connecting strips detachably joined to the bases;
wherein the bases respectively correspond to portions of the head of the living body suitable for extracting brain activity signals of interest when the assembled head-mounted module is worn on the head of the living body.

2. The assembled head-mounted module of claim 1, wherein each of the bases has at least one joining structure adapted to be joined with at least one of the first connecting strips.

3. The assembled head-mounted module of claim 2, wherein the at least one joining structure of each of the bases comprises a joining protrusion, a slot, a female buckle, or a combination thereof.

4. The assembled head-mounted module of claim 1, wherein each of the first connecting strips has at least one joining structure adapted to be joined with at least one of the bases.

5. The assembled head-mounted module of claim 4, wherein the at least one joining structure of each of the first connecting strips comprises a joining hole, a tongue, a male buckle, or a combination thereof.

6. The assembled head-mounted module of claim 1, wherein a material of the bases and the connecting structures is plastic, resin, or a combination thereof.

7. The assembled head-mounted module of claim 1, wherein the first connecting strips are flexible strips, bendable strips, length-adjustable strips, or a combination thereof.

8. The assembled head-mounted module of claim 1, further comprising:
at least one second connecting strip detachably joined to at least one of the first connecting strips;
wherein the at least one second connecting strip corresponds to at least one ear portion of the living body when the assembled head-mounted module is worn on the head of the living body.

9. The assembled head-mounted module of claim 8, wherein the at least one second connecting strip is two second connecting strips, and the second connecting strips respectively correspond to two opposite ear portions of the living body when the assembled head-mounted module is worn on the head of the living body.

10. The assembled head-mounted module of claim 9, further comprising:
a third connecting strip detachably joined to the second connecting strips;
wherein the third connecting strip corresponds to a neck or a jaw of the living body when the assembled head-mounted module is worn on the head of the living body.

11. The assembled head-mounted module of claim 10, wherein the third connecting strip is a length-adjustable strip or an elastic strip.

12. The assembled head-mounted module of claim 8, wherein the at least one second connecting strip has a ring shape or has an arc portion.

13. A head-mounted brain activity sensing device adapted to be worn on a head of a living body and comprising:
a plurality of first bases;
a plurality of first connecting strips detachably joined to the first bases; and
one or more signal extractors respectively removably placed in the first bases and configured to extract brain activity signals;
wherein the one or more signal extractors respectively correspond to one or more portions of the head of the living body suitable for extracting brain activity signals when the head-mounted brain activity sensing device is worn on the head of the living body.

14. The head-mounted brain activity sensing device of claim 13, further comprising:
one or more conductive lines respectively coupled to the one or more signal extractors.

15. The head-mounted brain activity sensing device of claim 14, wherein each of the one or more conductive wires is at least partially encapsulated or covered by the first bases or the first connecting strips.

16. The head-mounted brain activity sensing device of claim 14, further comprising:
a second base detachably joined to one or more of the first connecting strips; and
a breakout box removably placed in the second base and coupled to the one or more conductive wires.

17. The head-mounted brain activity sensing device of claim 16, wherein the second base corresponds to a posterior portion of the living body when the wearable physical monitoring apparatus is worn on the head of the living body.

18. The head-mounted brain activity sensing device of claim 13, wherein each of the one or more signal extractors comprises a sensor, a probe, or a combination thereof.

19. The head-mounted brain activity sensing device of claim 13, further comprising:
one or more stimulators removably placed in the first bases, the one or more stimulators configured to perform brain activity stimulation on the head of the living body.

20. The head-mounted brain activity sensing device of claim 19, wherein one of the one or more signal extractors and one of the one or more stimulators are placed in the same one of the first bases.
